(19) United States
(12) Patent Application Publication
(10) Pub. No.: US 2022/0143433 A1
(43) Pub. Date: May 12, 2022

(54) Surgical Face Shield

(71) Applicant: Wisconsin Alumni Research Foundation, Madison, WI (US)

(72) Inventors: Michael Bentz, Madison, WI (US); Lennon Rodgers, Madison, WI (US); Karl Williamson, Baltimore, MD (US)

(21) Appl. No.: 17/509,600

(22) Filed: Oct. 25, 2021

Related U.S. Application Data
(60) Provisional application No. 63/110,652, filed on Nov. 6, 2020.

(51) Int. Cl. A62B 18/08; A41D 13/11; A61F 9/02

(52) U.S. Cl. CPC A62B 18/082; A41D 13/1184; A61F 9/026; A61F 9/028; A61F 9/029

(57) ABSTRACT

A face shield is formed of a sheet of flexible material bent in an arc about a user's face with ends supported by the temples of glasses worn by the user. Cut outs allow use of the face shield without interference with the glasses lenses or accessories such as eye loupes worn during

### CROSS REFERENCE TO RELATED APPLICATION

This application claims the benefit of U.S. provisional application 63/110,652 filed Nov. 6, 2020 and hereby incorporated by reference.

The present invention relates to face shields of the type intended to reduce or prevent infection and in particular to a face shield that adapts well for surgeons wearing glasses supporting loupes or the like.

The importance of face shields in preventing the transmission of infectious agents is increasingly well established. Typically, a face shield will include a headband that can be clamped about the head to support a transparent shield that can be positioned over the eyes and mouth of the user. Such face shields can be relatively expensive and require complex tooling for manufacture, placing strains on the supply system, for example, in situations where there are sudden high demands for face shields such as pandemics or the like and new manufacturing sources are required. Standard face shields can also interfere with eye loupes commonly worn during surgery.

### SUMMARY OF THE INVENTION

The present invention provides a face shield comprised of a thin sheet of material adapted to curve about the face and be supported on the temple portions of eyeglasses. By eliminating the need for a headband, a difficult to sterilize and expensive component is eliminated and the remaining structure may be shipped flat at high density and low cost. The shield includes a cut out around the lenses of the glasses eliminating interference with vision and with the protruding structure of eye loupes and the like, while being stabilized against the lenses.

Specifically, the invention provides a face shield for attachment to eyeglasses and having a resilient flexible sheet with an upper edge and left and right edges and adapted to flex in an arc over a front of the user's face and rearwardly along the temples of the eyeglasses when attached to the temples of the eyeglasses. The flexible sheet may provide a pair of openings at each of left and right positions on the sheet, respectively, proximate to the left and right edge and upper edge of the sheet through which temples of the eyeglasses may be threaded to retain the left and right portions against the temples. A cutout is positioned beneath the upper edge and between the left and right portions of the sheet over the lenses of the glasses to provide an upper cut out edge fitting in front of and passing along a lower edge of the lenses.

It is thus a feature of at least one embodiment of the invention to provide a low-cost face shield that can be shipped and stored in a high density configuration without interference from clips, headbands, or the like. It is another feature of at least one embodiment of the invention to provide a face shield that can be used with glasses and glasses attachments such as eye loupes without interference from distortion by the material of the shield or from light reflection.

The sheet may further include a breath bypass vent positioned below the upper cut out edge and aligned beneath the bridge portion of the glasses.

It is thus a feature of at least one embodiment of the invention to provide a shield that can fit closely against protective eyewear without undue fogging of the lenses of the eyeglasses. The breath bypass vent provides a path of diffusion of moist air out from behind the shield while still maintaining the protective shield properties.

The breath bypass vent may be an opening extending downwardly away from the upper cut out edge aligned with the bridge portion of the glasses.

It is thus a feature of at least one embodiment of the invention to provide a simple vent structure consistent with the high-density storage and shipping properties of the face shield.

The sheet may further include guidelines positioned around the breath bypass vent defining cut lines to remove additional material to increase the size of the breath bypass vent.

It is thus a feature of at least one embodiment of the invention to permit on-site customization of the breath bypass vent for improved operation.

The guidelines may be perforations promoting a separation of the flexible sheet along the perforations.

It is thus a feature of at least one embodiment of the invention to allow customization of the breath bypass vent without the need for cutting tools or the like.

The pairs of openings may be vertically extending slots.

It is thus a feature of at least one embodiment of the invention to provide openings for receiving the glasses temples that accommodate temples of different sizes, are easy to thread, and still maintain good frictional contact.

The resilient flexible sheet may have a perimeter that fits within an 8.25×11.5″ rectangle.

It is thus a feature of at least one embodiment of the invention to permit the use of feedstock in standard letter or A4 sizes and/or storage and shipping in containers intended for such materials.

The resilient flexible sheet may be transparent.

It is thus a feature of at least one embodiment of the invention to maintain visibility of the user's face for improved communication and patient assurance.

The resilient flexible sheet may have a thickness of less than 0.01 inches and/or may be a polymer selected from the group consisting of polyester, PET, PETG, and polycarbonate.

It is thus a feature of at least one embodiment of the invention to provide a design that can work with light-weight, readily available, and low-cost materials.

The sheet may include an anti-fog coating on at least one surface of the resilient flexible sheet.

It is thus a feature of at least one embodiment of the invention to reduce the accumulation of condensation on the shield such as could drip into the surgical field.

These particular objects and advantages may apply to only some embodiments falling within the claims and thus do not define the scope of the invention.

### BRIEF DESCRIPTION OF THE DRAWINGS

FIG. 1 is a perspective view of the face shield of the present invention during use as supported by a surgeon's eyeglasses, the latter holding eye loupes and showing the fitting of the shield about the face of the user as supported on the glasses temples and overlapping a front lower edge of the eyeglass lenses;

FIG. 2 is a flattened depiction of the shield of FIG. 1 showing the various cutouts and dimensions of a preferred embodiment;

FIGS. 3a and 3b are fragmentary detailed views of alternative embodiments of a breath bypass vent serving to reduce fogging of glasses when the shield is used as shown in FIG. 1; and

FIG. 4 is a depiction of airflow paths past the shield showing the breath bypass vent's channeling of breath away from the lens surfaces to reduce fogging.

### DETAILED DESCRIPTION OF THE PREFERRED EMBODIMENT

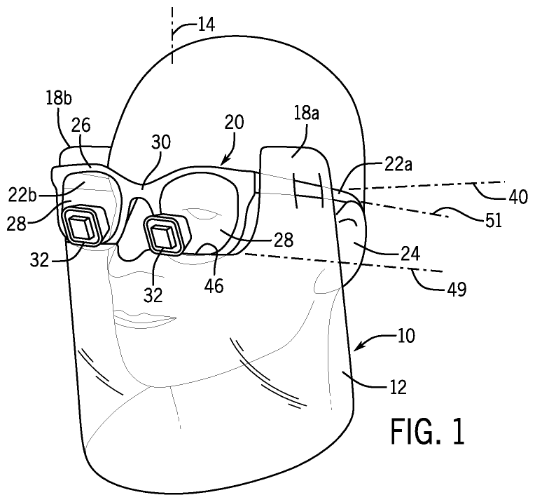

Referring now to FIG. 1, the present invention provides a face shield 10 formed of a thin sheet 12 of flexible material that may be curved in an arc about a vertical axis 14 to cover the nose and mouth of a user 16. The sheet 12 in a relaxed state may lie flat and curvature may be maintained by attachment tabs 18a and 18b positioned at right and left sides on the upper edge of the sheet 12 attachable to glasses 20 worn by the user 16.

The glasses 20 may be standard prescription glasses, safety glasses, or eye-loupe glasses or the like. The glasses 20 provide rigid left and right temples 22a and 22 extending along sides of the user's head to distal ends fitting over the left and right ears 24 of the user. Proximal ends of the temples attach to left and right sides of a lens frame 26 holding lenses 28 positioned over the left and right eyes of the user to be viewed therethrough. Between the lenses 28, the lens frame 26 provides a bridge 30 generally positioned over the nose of the user and including supports (not shown) for supporting the glasses 20 on an upper part of the user's nose. Eye loupes 32, for example, used by surgeons and the like to provide a magnified field-of-view, may be attached to or engaged with the front surfaces of the lenses extending outwardly from the front surface of the lenses 28 of the glasses 20 away from the face of the user 16.

The sheet 12, may be a polymer film, for example, having a thickness of 0.007 inches and desirably less than 0.01 inch and a total area fitting within the dimensions of an 8.25×11″ rectangle. In this respect, the present invention can be constructed of or printed on standard letter sized or A4 material. In one embodiment the sheet 12 is transparent. The sheet 12 may be constructed from a variety of different materials including, for example, PET (polyethylene terephthalate), PETG (polyethylene terephthalate glycol), polycarbonate and the like. One or both surfaces of the sheet 12 may be treated with an anti-fog agent to reduce the formation of condensation on the sheet.

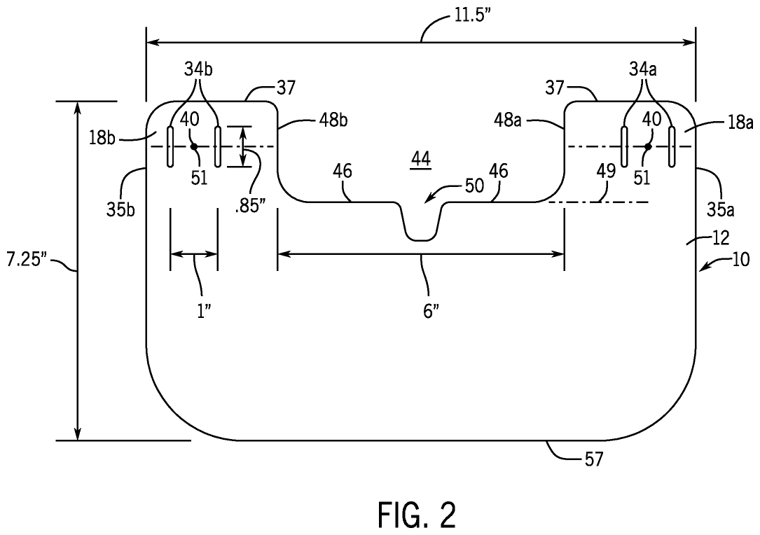

Referring now also to FIG. 2, the attachment tabs 18 of sheet 12 may each include one pair of horizontally separated, vertically oriented slots 34a and 34b. The slots 34 are positioned near left and right vertical edges 35a and 35b of the sheet 12 and near an upper edge 37 of the sheet 12 so that the glasses temples 22a and 22b, respectively, may be fed through the slots to attach the tabs 18 to the respective temples when the sheet 12 is properly curved as shown in FIG. 1. In particular, the temples 22 may be threaded through the slots 34 so that the temples 22 are exposed on an outside (with respect to the user 16) surface of the sheet 12 between the slots 34. Generally the slots may have a separation of at least ½ inch horizontally and a height of greater than ½ inch vertically, the latter to provide ample clearance and flexibility for inserting the temples 22 through the slots 34. The narrowness of the slots 34 and the stiffness of the material of the sheet 12 provide resistance to torsion caused by weight of the shield 10 about a horizontal axis 40 passing through a center point between the slots of each pair of slots 34. The vertical extent of the slots 34 accommodates different styles of glasses where the temples attach to the upper edge of the frame 26 or to a side edge of the frame 26. The slots 34 terminate in hemi circles to eliminate stress points that could lead to tears in the sheet 12.

When the style of the glasses 20 is known, and in particular the height of the temple 22 with respect to the lower edges of the lenses 28, resistance against rotation of the shield 10 about axis 40 can be limited by the lower edge of the rearmost slot 34 and the upper edge of the front most slot 34 abutting the temple 22. In order to maintain slot height for easy threading of the temple 22 through the slots 34, in this case the slots 34 may be vertically staggered to more closely align the lower edge of the rearmost slot and upper edge of the front most slot along a temple axis 51.

Referring still to FIG. 2, the attachment tabs 18 extend upward on either side of a central cut out region 44 below the upper edges 37 of the sheet 12. This central cut out region 44 is substantially centered between the attachment tabs 18 and produces a generally horizontal cut out edge 46 positioned below the upper edge 37 of the sheet and the temple axis 51. The height of this cut out edge 46 is such that the cut out edge 46 extending along axis 49 passes along a lower edge of the outer surface of the lenses 28 of the glasses 20 when the shield 10 is installed on the glasses 20. As so positioned, the front surface of the lenses 28 blocks rearward motion of the shield 10 of the glasses 20. As such the cut out edges 46 may be captured against a front surface of the lens 28 and below the outwardly projecting eye loupes 32 to be located thereby. While generally lightweight, sheet 12 will be amply supported by friction or abutment between the slots 34 and the temple 22, and it will be appreciated that a rear surface beneath the cut out edge 46 may include adhesive (for example, protected by a release liner) or flexible cutouts that engage the circumference of the eye loupes 32 or the like for further stability.

The width of the cut out edge 46, defined on the left and right by opposed vertical edges 48a and 48b defining opposed inner sides of the tabs 18, will be such as to extend beyond the width of the lens frame 26 so that these vertical edges 48 are spaced from the lens frame 26 to curve about the temples 22.

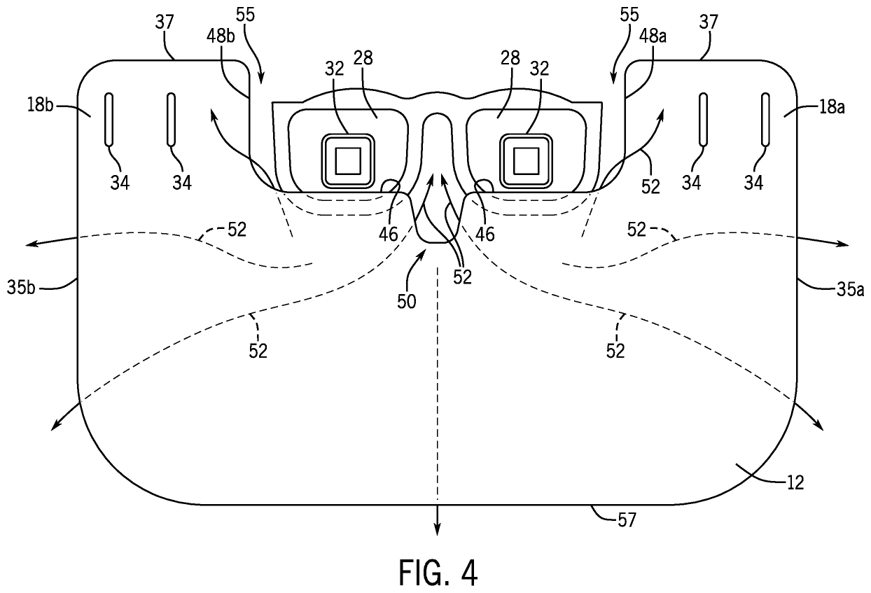

Referring also to FIG. 4, a breath bypass vent 50 is positioned midway between the vertical edges 48 along the cut out edge 46. In one embodiment the breath bypass vent 50 provides a downwardly extending notch position to be vertically aligned with and beneath the bridge 30 of the glasses 20. At this location, the breath bypass vent 50 promotes a conduction of exhalation air 52 away from the lenses 28 and upward along the bridge 30 to diffuse upwardly and outwardly away from the lenses 28 to reduce fogging. This conduction is in addition to conduction around the lenses 28 gaps 55 between the lenses 28 and the vertical edges 48 and conduction downward and outward past edges 35 and lower edge 57 of the sheet 12. In this way moist air is encouraged to bypass the lenses 28 to reduce fogging thereof.

Generally, the edges 35 curve about inwardly in an arc having a several inch radius to join with lower edge 57 to eliminate sharp corners that may snag or catch clothing or equipment. The height of the lower edge 57 is set to allow the user to look downward at approximately 45 degrees without interference between lower edge 57 and the user's neck.

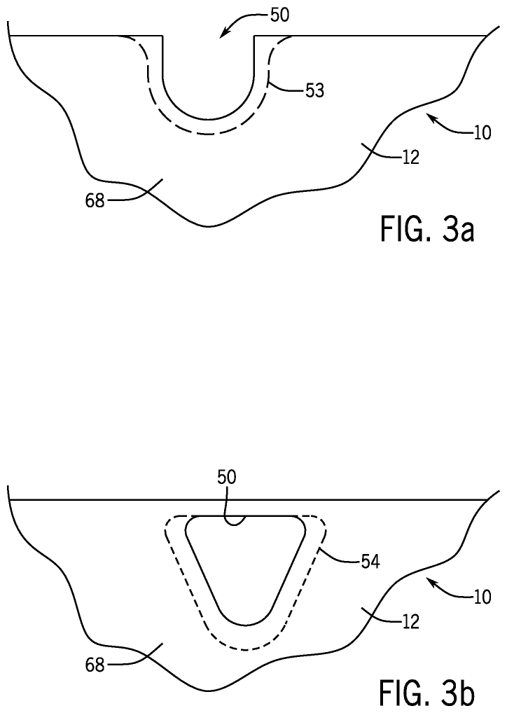

Referring now to FIG. 3a, the breath bypass vent 50, in a notch form as discussed above, will generally be sized and positioned to be displaced from the upper bridge of the nose of the user as spaced by contact between a tip of the nose and the sheet 12 at a contact region 60 displaced below the breath bypass vent 50.

Such spacing is also provided by the embodiment shown in FIG. 3b where the breath bypass vent 50 is not a notch but a similarly positioned hole providing suitable opening providing the necessary airflow. Printed cut lines 53 (FIG. 3a) or perforations 54 (FIG. 3b) may provide guidelines around the breath bypass vent 50 to allow the vent size to be enlarged by the user as necessary (for example, by cutting with scissors or separating at the perforations) to reduced glasses fogging.

It will be appreciated that the shield 10 of the present invention may be shipped flat, for example, as separated with paper divider sheets or the like in high density packages of many identical shields 10, greatly reducing otherwise substantial costs in transportation and storage of otherwise bulky face shields. The paper divider sheets may also be printed with instruction material and, for example, guidelines 52 in lieu of those shown above with respect FIG. 3a. The sheets 12 may be constructed, for example, by die cutting or laser cutting or the like allowing them to be fabricated either in mass production or locally at smaller scale. in addition, the sheets 12 may be fabricated in one operation (e.g. die cutting) of the meeting assembly concerns and increasing production rate. The ability to ship and store the shields flat also may assist in the generation of simple dispensing systems that can store and dispense such flat sheets while maintaining a sterile environment. Importantly, the use of slots prevents the shield from being dislodged (as might occur with clips) and falling into a sterile operative field.

As used herein, the shield 10 may be considered the sheet 12 in isolation or the combination of the sheet 12 and the glasses 20 as will be evident from context. Although not depicted, it is contemplated that the user 16 will also be wearing a surgical mask covering the mouth and nose.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

While the present invention is particularly useful for surgeons wearing eyeglasses with loupes, it will also be appreciated that has benefits for individuals wearing headlights or head mounted cameras including dentists and other healthcare workers.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A face shield for attachment to eyeglasses of a type wearable by a user to provide front facing lenses positioned in front of the user's eyes and separated by a bridge portion separating the lenses and positioned over a user's nose, the lenses flanked on left and right sides by rearwardly extending temples sized to pass over and rest on a user's ears, the face shield comprising:
a resilient flexible sheet having an upper edge and left and right edges and adapted to flex in an arc over a front of the user's face and rearwardly along the temples of the eyeglasses when attached to the temples and as worn by the user, the flexible sheet further providing:
(a) a pair of openings at each of left and right positions on the flexible sheet, respectively, the positions proximate to the left and right edges and upper edge of the flexible sheet, the openings through which temples of the eyeglasses may be threaded to retain the left and right portions against the temples; and
(b) an opening positioned beneath the upper edge and between the left and right portions over the lenses to provide an upper cut out edge fitting in front of and passing along a lower edge of the lenses.

2. The face shield of claim 1 further including a breath bypass vent positioned below the upper cut out edge and aligned beneath the bridge portion of the glasses.

3. The face shield of claim 2 wherein the breath bypass vent and opening extend downwardly away from the upper cut out edge aligning with the bridge portion of the glasses.

4. The face shield of claim 2 wherein further including guidelines positioned around the breath bypass vent defining cut lines to remove additional material to increase the size of the breath bypass vent.

5. The face shield of claim 4 wherein the guidelines are perforations promoting a separation of the flexible sheet along the perforations.

6. The face shield of claim 1 wherein in the pairs of openings are vertically extending slots.

7. The face shield of claim 1 wherein the resilient flexible sheet has a perimeter that fits within an 8.25×11.5″ rectangle.

8. The face shield of claim 1 wherein the resilient flexible sheet is transparent.

9. The face shield of claim 1 wherein the resilient flexible sheet has a thickness of less than 0.01 inches.

10. The face shield of claim 1 wherein the resilient flexible sheet is a polymer selected from the group consisting of polyester, PET, PETG, and polycarbonate.

11. The face shield of claim 1 further including an anti-fog coating on at least one surface of the resilient flexible sheet.

12. The face shield of claim 1 including the glasses and wherein the glasses provide eye loupes extending forwardly from front surfaces of lenses of the glasses.
